(19) United States
(12) Patent Application Publication
(10) Pub. No.: US 2014/0207213 A1
(43) Pub. Date: Jul. 24, 2014

(54) LASER METHOD FOR TREATING HYPERHIDROSIS TARGETING SWEAT GLANDS

(71) Applicant: Khalilullah A. Khatri, Lexington, MA (US)

(72) Inventor: Khalilullah A. Khatri, Lexington, MA (US)

(21) Appl. No.: 14/158,952

(22) Filed: Jan. 20, 2014

Related U.S. Application Data
(60) Provisional application No. 61/755,568, filed on Jan. 23, 2013.

(51) Int. Cl. A61N 5/06

(52) U.S. Cl. CPC A61N 5/0616
USPC 607/89

(57) ABSTRACT

A non-surgical, drug-free hyperhidrosis treatment includes applying infra-red laser irradiation having a wavelength of at least approximately 1064 nm to a plurality of discrete spots in the affected area. The operating parameters of the infra-red laser are adjusted so that the irradiation penetrates the skin at each spot and deactivates the underlying sweat glands. The degree of sweat suppression is controlled by adjusting the number of and spacing between the spots. The laser light can be generated by a NdYAG laser. The spot size can be approximately 2 mm, the pulse duration approximately 0.65 ms, and the laser power approximately 9300 Watts. A prior art laser source can be used to manually implement the invention, or a special purpose apparatus can automatically deflect the infra-red beam under digital control between the desired spot locations. In embodiments, the controller further controls the laser timing and other operating parameters.

### RELATED APPLICATIONS

This application claims the benefit of U.S. Provisional Application No. 61/755,568, filed Jan. 23, 2013, which is herein incorporated by reference in its entirety for all purposes.

### FIELD OF THE INVENTION

The invention relates to methods of treating skin pathologies, and more particularly, to methods for treating hyperhidrosis.

### BACKGROUND OF THE INVENTION

Hyperhidrosis is a condition characterized by abnormally increased perspiration, in excess of that required for regulation of body temperature. Hyperhidrosis can either be generalized or localized to specific parts of the body. Hands, feet, armpits, and the groin area are among the most active regions of perspiration, due to the relatively high concentration of sweat glands in those regions. When excessive sweating is localized, it is referred to as primary or focal hyperhidrosis.

Hyperhidrosis can be embarrassing, offensive to others, and detrimental to the health of the affected area(s). Antiperspirants can be helpful, but are often insufficient. Special aluminum chloride based compounds can be applied to temporarily clog sweat glands, but this approach can be messy, does not always work (especially on palms), can cause skin irritations, and is a temporary solution at best.

Drug therapies are available, such as anticholinergics, which interfere with a chemical that goes between the nerve and sweat gland. However, drugs are non-specific, and block that chemical everywhere in the body, so that perspiration can be reduced to below-desirable levels in areas that are not affected by hyperhidrosis. Also, drugs may be undesirable due to side effects, such as dry eyes, dry mouth, blurred vision, and urinary retention.

Botox is sometimes applied to the skin so as to paralyze the nerves that transmit messages to the sweat glands. FIG. 1 illustrates a result of botox treatment applied only to right palm 100 of a patient as a treatment for hyperhidrosis. After the treatment, an iodine starch test was performed, whereby an iodine solution was applied to both palms of the patient, and after the iodine solution dried starch was sprinkled on the areas. The starch-iodine combination turned a dark blue color wherever there was excess sweat. Reduction of sweat due to botox application can be seen from the lighter color of the right palm 100 as compared to the darker color of the left palm 102. However, this approach requires anesthetizing of the affected area, and sometimes general anesthesia, followed by 20 to 50 injections in various areas. The treatments are painful, and can cause significant weakness in the affected areas. At best, Botox is a temporary solution, and must be repeated every 4-8 months.

Surgical approaches are also available. Arthroscopic axillary gland excision can be performed to treat underarm hyperhidrosis. A target area is mapped out in the underarm, and then a tiny incision is made, the size of a dime or smaller. A small arthroscopic shaver is then inserted through this hole and passed back and forth to destroy or remove all the sweat glands in that area right under the skin. Liposuction can also be used for underarm hyperhidrosis. For hyperhidrosis in other regions of the body, such as the palms and feet, thoracascopic sympathectomy (“ETS”) can be used to interrupt the nerve impulses that trigger sweating. These surgical methods are relatively permanent solutions to hyperhidrosis, but of course surgery always involves risks of infection and adverse reaction to anesthetics, and is usually considered a last resort.

Iontophoresis is a non-surgical, non-drug approach where an electrical current is periodically applied to the affected areas, such as the palms or feet, to paralyze or inactivate sweat glands. However, this approach is only successful about half of the time.

For underarm hyperhidrosis, a non-invasive hand-held device can be used to deliver controlled electromagnetic energy to an area of the skin that is affected by hyperhidrosis, resulting in heating and decomposition of the sweat glands. This approach is highly effective and essentially permanent, but it requires a fat layer underlying the skin that can absorb the electromagnetic energy, and so is limited mainly to the underarms.

What is needed, therefore, is a non-surgical, drug-free technique for treating hyperhidrosis in virtually any affected area, preferably with long-lasting effects.

### SUMMARY OF THE INVENTION

The present invention is a non-surgical, drug-free method for treating hyperhidrosis in virtually any affected area. According to the present method, infrared laser irradiation is applied to the affected area, penetrating the skin and inactivating the underlying sweat glands. The infra-red irradiation is applied in small spots on the skin, so that the degree of sweat suppression can be controlled by controlling the density of the treated spots. Discomfort is tolerable for most patients, so that anesthesia is not usually required. In embodiments, treatments are repeated once per week for a series of approximately six treatments, resulting in some cases in an 80% reduction of perspiration.

Before the present invention, laser irradiation was not considered to be a candidate for treating hyperhidrosis, because laser light in general does not penetrate skin deeply enough to affect the underlying sweat glands. However, infra-red light of approximately 1064 nm wavelength or longer penetrates skin more deeply than light at shorter wavelengths, and is able to reach the sweat glands directly. No underlying fat layer is required.

In embodiments, the applied irradiation is pulsed 1064 nm laser light emitted by an NdYAG laser. In some of these embodiments, the laser power is approximately 9300 Watts, the dosage at each spot has a fluence of 191 J/cm², the spot size is about 2 mm diameter, the laser pulse duration is approximately 0.65 ms, and/or the spots are about 1 cm apart.

Some skin lightening and/or other discoloration may occur, which can be a concern when treating facial hyperhidrosis. However, any such effects are mainly inconsequential for other treated regions. Of course, as with any high power laser method, there is also a risk of eye damage if the laser irradiation should somehow be misdirected into someone's eyes, but this can easily be avoided if common laser safety standards are followed. There are essentially no other side effects or risks.

An apparatus of the present invention includes a laser light source and a processor-controlled beam directing mechanism that applies the light pulses to a plurality of spots in the affected area at a desired power, duration, spot size, and application pattern. In embodiments the laser light source is a pulsed NdYAG laser. Some embodiments include a surrounding shield that helps to position and maintain the device over the affected area and also isolates and protects the operator and patient from any scattered laser light.

One general aspect of the present invention is a method for treating hyperhidrosis on an affected area of a patient's skin. The method includes applying infra-red laser irradiation generated by an infra-red laser to a discrete spot within the affected area, the infra-red laser irradiation having a wavelength of about approximately 1064 nm or longer, the irradiation having sufficient power, duration, and concentration to penetrate the patient's skin in the discrete spot and irradiate any underlying sweat glands, thereby deactivating the irradiated sweat glands, redirecting the infra-red laser irradiation to another discrete spot within the affected area, and repeating the steps of applying and redirecting until a desired number of discrete spots within the affected area have been irradiated.

In embodiments, the infra-red laser is a NdYAG laser. In some embodiments, the discrete spots are arranged approximately as a grid of equally spaced spots. In various embodiments, the discrete spots are separated from each other by a distance of approximately one centimeter.

In certain embodiments the diameter of each discrete spot is approximately 2 mm. In other embodiments the infra-red laser irradiation is applied at a power of approximately 9300 Watts.

In embodiments, the infra-red laser irradiation is applied in bursts lasting approximately 0.65 ms.

In various embodiments, the infra-red laser irradiation is automatically directed from each discrete spot to the next by a beam deflector controlled by a deflection controller, so as to apply the irradiation to discrete spots in the affected area in a desired pattern. And in some of these embodiments the deflection controller further controls at least one of an output power, pulse duration, and pulse timing of the infra-red laser.

A second general aspect of the present invention is an apparatus for treating hyperhidrosis on an affected area of a patient's skin. The apparatus includes a beam deflector, a laser light input, configured to deliver laser light from an infra-red laser to the beam deflector, and a controller programmed to vary the orientation of the beam deflector so that the laser light is applied to a series of discrete spots within the affected area of the patient's skin, each of the discrete spots thereby receiving a laser irradiation dosage of a desired power, spot size, and duration.

Embodiments further include an open-ended, opaque nozzle surrounding the beam deflector and laser light input, and configured so that the infra-red laser irradiation is directed through the open end of the nozzle by the beam deflector.

In some embodiments, the controller further controls a beam output timing of the infra-red laser. In some of these embodiments the controller further controls an output power of the infra-red laser. And in other of these embodiments the controller further controls a spot size of the infra-red laser irradiation.

The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### BRIEF DESCRIPTION OF THE DRAWINGS

FIG. 1 is a photograph of a patient's palms, where the right palm has been treated for hyperhidrosis using botox, and the left palm has not been treated, an iodine-starch test having been applied to both palms to indicate a degree of residual excess perspiration;

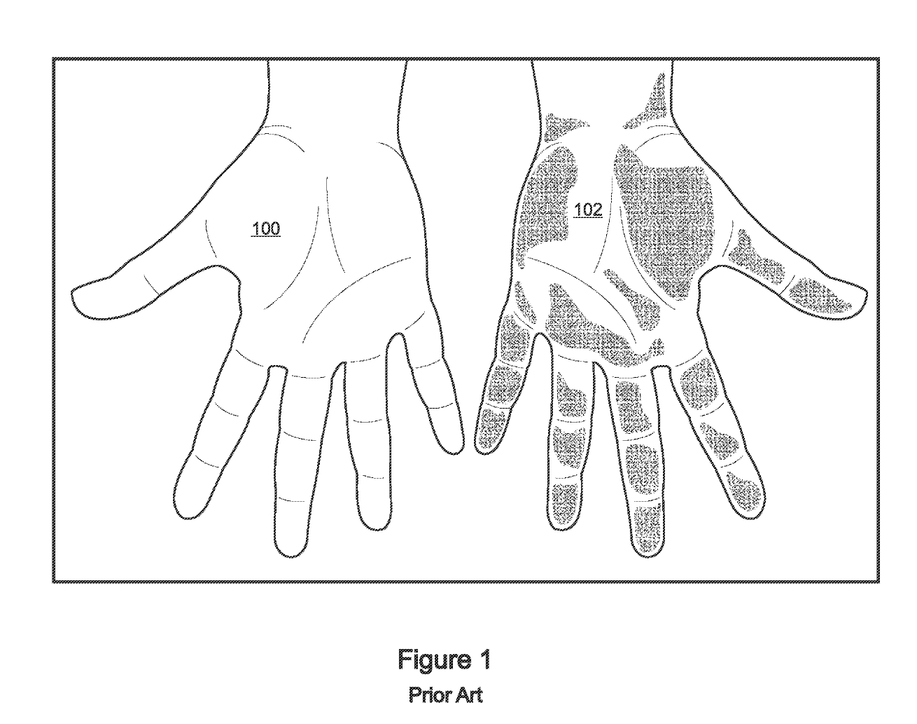

FIG. 2 is a cross sectional diagram of skin anatomy;

FIG. 3 is a chart illustrating the depths to which laser light generated by different types of laser will penetrate into skin as a function of increasing laser wavelength;

FIG. 4 is a view of a patient's palm showing a pattern of discrete spots where the infra-red laser treatment of the present invention is applied in an embodiment of the present invention;

FIG. 5 is a photograph of a prior art infra-red laser source that can be used to manually implement the method of the present invention;

FIG. 6 is a perspective view of an apparatus configured to automatically implement the present invention in an embodiment; and

FIG. 7 is a flow diagram indicating the steps of the present invention in an embodiment.

### DETAILED DESCRIPTION

The present invention is a non-surgical, drug-free method for treating hyperhidrosis in virtually any affected area. According to the method, infrared laser irradiation is applied to the affected area, which penetrates the skin and inactivates the underlying sweat glands.

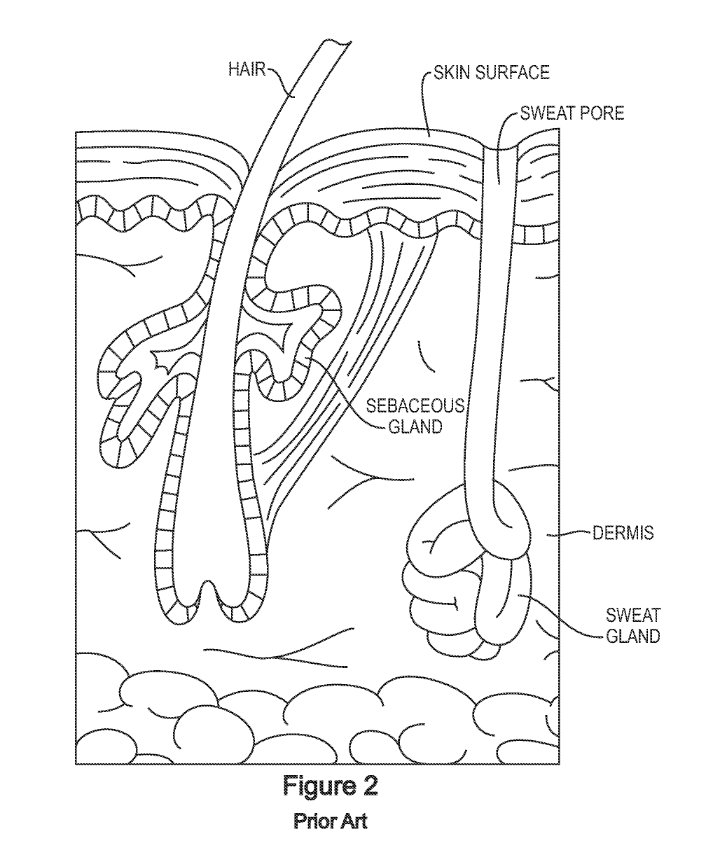

FIG. 2 is a cross-sectional illustration of skin anatomy. As can be seen from the figure, the sweat gland is positioned well below the surface of the skin. Before the present invention, laser irradiation was not considered to be a candidate for treating hyperhidrosis, because laser light at most wavelengths does not penetrate skin deeply enough to affect the underlying sweat glands.

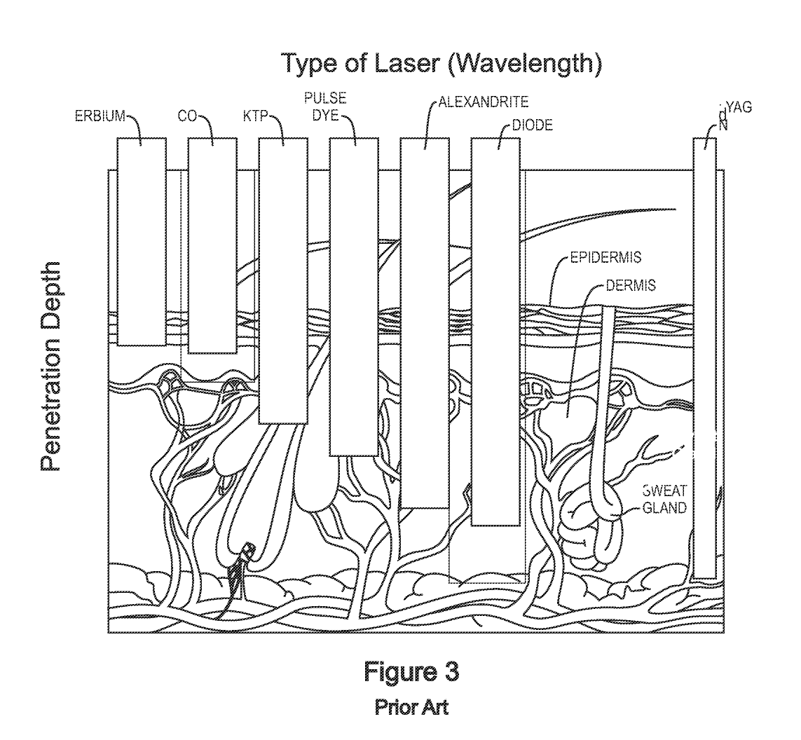

However, infra-red light of approximately 1064 nm wavelength or longer penetrates skin more deeply than light at shorter wavelengths, and is able to reach the sweat glands directly. No underlying fat layer is required. FIG. 3 illustrates skin penetration depths for different laser wavelengths. It can be seen from the figure that a relatively long wavelength, such as is produced by a NdYAG laser, is required to penetrate skin deeply enough to reach and irradiate the sweat glands.

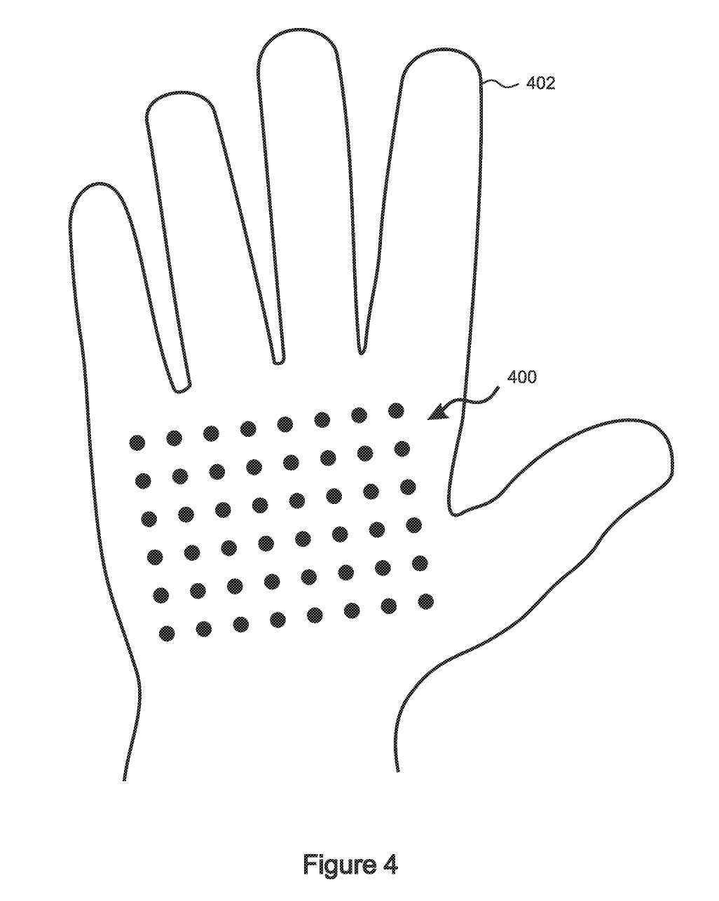

In embodiments, the applied irradiation is pulsed 1064 nm laser light emitted by an NdYAG laser. In some of these embodiments, the laser power is approximately 9300 Watts, the dosage at each spot has a fluence of 191 J/cm², the spot size is about 2 mm diameter, and/or the laser pulse duration is approximately 0.65 ms. With reference to FIG. 4, in various embodiments the laser is applied to a plurality of discrete spots in the affected area, which can be spaced about 1 cm apart. FIG. 4 illustrates a pattern of discrete laser spots 400 applied to the palm of a patient's hand 402. The spots are indicated as dark black circles for clarity of illustration, but in reality there is little if any skin discoloration.

Some skin lightening and/or other discoloration may occur due to implementation of the present invention, which can be a concern when treating facial hyperhidrosis. However, any such effects are mainly inconsequential for other treated areas. Of course, as with any high power laser method, there is also a risk of eye damage if the laser irradiation should somehow be misdirected into someone's eyes, but this can easily be avoided if common laser safety standards are followed. There are essentially no other side effects or risks associated with the present invention.

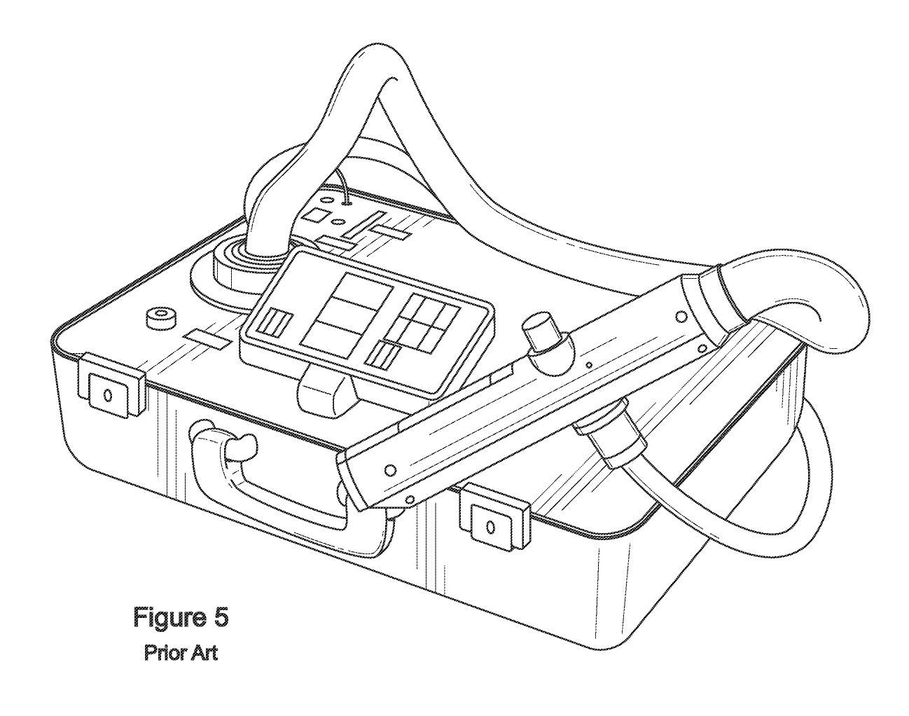

In some embodiments, the present invention is implemented using NdYAG laser light produced by a standard laser source such as the one shown in FIG. 5. The surgeon directs the laser from one spot location to the next, and triggers bursts of irradiation having the desired power, duration, and spot size.

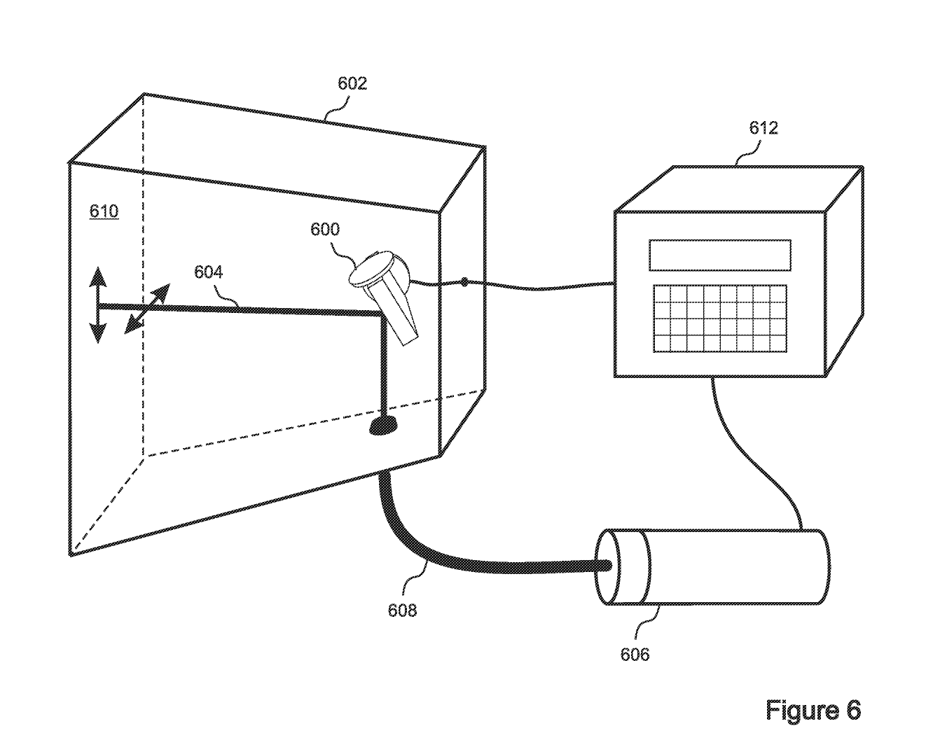

With reference to FIG. 6, in other embodiments the invention includes a special apparatus that is used to efficiently implement the method of the present invention. A beam deflector 600 is mounted within an opaque, open-ended nozzle 602. Laser light 604 from an NdYAG laser 606 is delivered to the nozzle 602 by a fiber optic cable 608, and the light 604 is reflected from the beam deflector 600 toward the open end 610 of the nozzle, which is placed against the affected area. The opaque nozzle 602 thereby provides enhanced protection against eye damage by preventing any primary or scattered light from reaching the eyes of the operator or the patient. A controller 612 controls both the direction of the beam deflector 600 and the pulse timing of the laser 606 so as to direct the beam sequentially through a grid of discrete spot locations, and so as to apply laser irradiation to each discrete spot with a desired power, spot size, and duration.

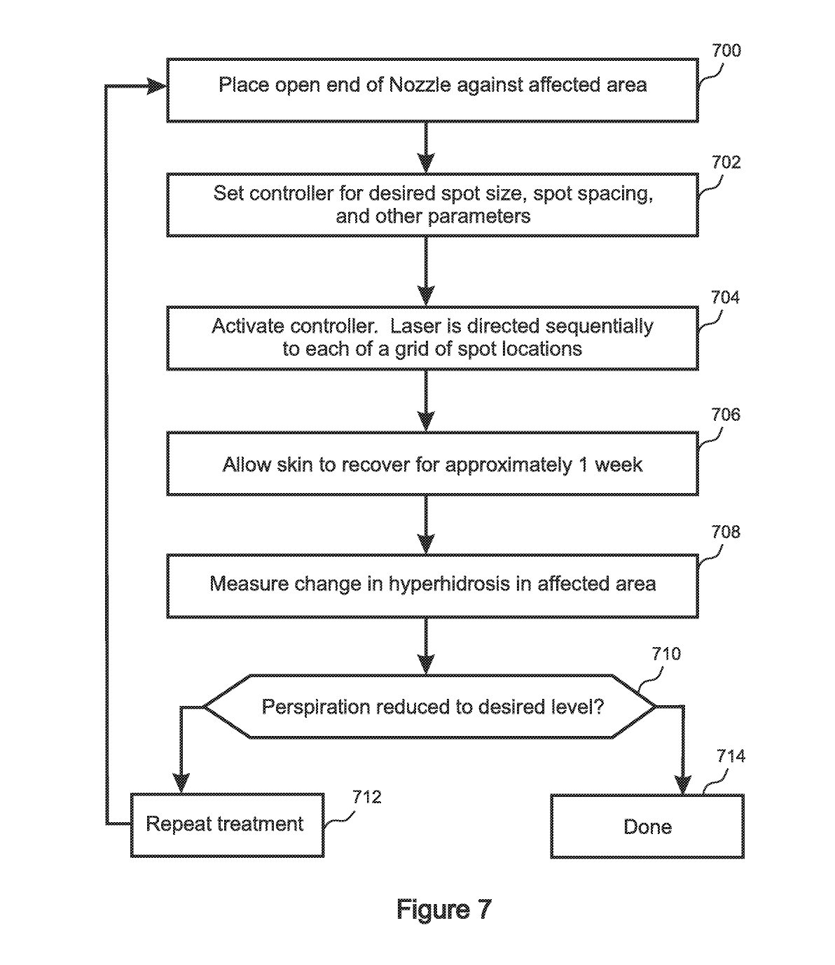

With reference to FIG. 7, to implement the present invention a physician or technician needs only to place the open end 610 of the nozzle 602 against the affected skin area 700 and select the desired operating parameters 702 using the controller 612. The controller 612 is then activated 704, and the laser bursts 604 are automatically applied throughout the desired grid of discrete spots 400 in the affected skin area.

In various embodiments, the treated skin is allowed to recover 706, typically for about a week, after which the remaining degree of hyperhidrosis (if any) is determined 708, for example by using the iodine starch test, and a decision is made as to whether additional treatments are needed 710. If so, then the process is repeated 712. Otherwise, the treatment program is complete 714. Typically, the relief from hyperhidrosis is long lasting, if not permanent, but if symptoms do return the treatment program can be repeated as necessary.

The foregoing description of the embodiments of the invention has been presented for the purposes of illustration and description. It is not intended to be exhaustive or to limit the invention to the precise form disclosed. Many modifications and variations are possible in light of this disclosure. It is intended that the scope of the invention be limited not by this detailed description, but rather by the claims appended hereto.

What is claimed is:

1. A method for treating hyperhidrosis on an affected area of a patient's skin, the method comprising:
applying infra-red laser irradiation generated by an infra-red laser to a discrete spot within the affected area, the infra-red laser irradiation having a wavelength of about approximately 1064 nm or longer, the irradiation having sufficient power, duration, and concentration to penetrate the patient's skin in the discrete spot and irradiate any underlying sweat glands, thereby deactivating the irradiated sweat glands;
redirecting the infra-red laser irradiation to another discrete spot within the affected area; and
repeating the steps of applying and redirecting until a desired number of discrete spots within the affected area have been irradiated.

2. The method of claim 1, wherein the infra-red laser is a NdYAG laser.

3. The method of claim 1, wherein the discrete spots are arranged approximately as a grid of equally spaced spots.

4. The method of claim 3, wherein the discrete spots are separated from each other by a distance of approximately one centimeter.

5. The method of claim 1, wherein the diameter of each discrete spot is approximately 2 mm.

6. The method of claim 1, wherein the infra-red laser irradiation is applied at a power of approximately 9300 Watts.

7. The method of claim 1, wherein the infra-red laser irradiation is applied in bursts lasting approximately 0.65 ms.

8. The method of claim 1, wherein the infra-red laser irradiation is automatically directed from each discrete spot to the next by a beam deflector controlled by a deflection controller, so as to apply the irradiation to discrete spots in the affected area in a desired pattern.

9. The method of claim 8, wherein the deflection controller further controls at least one of an output power, pulse duration, and pulse timing of the infra-red laser.

10. An apparatus for treating hyperhidrosis on an affected area of a patient's skin, the apparatus comprising:
a beam deflector;
a laser light input, configured to deliver laser light from an infra-red laser to the beam deflector; and
a controller programmed to vary the orientation of the beam deflector so that the laser light is applied to a series of discrete spots within the affected area of the patient's skin, each of the discrete spots thereby receiving a laser irradiation dosage of a desired power, spot size, and duration.

11. The apparatus of claim 10, further including an open-ended, opaque nozzle surrounding the beam deflector and laser light input, and configured so that the infra-red laser irradiation is directed through the open end of the nozzle by the beam deflector.

12. The apparatus of claim 10, wherein the controller further controls a beam output timing of the infra-red laser.

13. The apparatus of claim 12, wherein the controller further controls an output power of the infra-red laser.

14. The apparatus of claim 12, wherein the controller further controls a spot size of the infra-red laser irradiation.
